(19) United States
(12) Patent Application Publication
(10) Pub. No.: US 2014/0356905 A1
(43) Pub. Date: Dec. 4, 2014

(54) Mammalian Cell Culture System for Large-Scale Expression of Recombinant Proteins

(71) Applicant: Rutgers, The State University of New Jersey, New Brunswick, NJ (US)

(72) Inventors: John D. Altman, Decatur, GA (US); John C. Shires, Stone Mountain, GA (US); Ankita Basant, Brookfield, CT (US); Jillian Whidby, Pilesgrove, NJ (US); Joseph Marcotrigiano, New Brunswick, NJ (US); Abdul G. Khan, New Brunswick, NJ (US)

(73) Assignee: Rutgers, The State University of New Jersey, New Brunswick, NJ (US)

(21) Appl. No.: 14/294,961

(22) Filed: Jun. 3, 2014

Related U.S. Application Data
(60) Provisional application No. 61/830,333, filed on Jun. 3, 2013.

(51) Int. Cl. C12N 15/86; C12P 21/00; C07K 14/705; C12N 7/00; C07K 14/005

(52) U.S. Cl. CPC C12N 15/86; C12N 7/00; C07K 14/005; C07K 14/70596; C12P 21/005; C12N 2740/15041; C12N 2740/15045; C12N 2770/24251; C12N 2501/999
USPC 435/69.1; 435/320.1; 435/369; 435/367

(57) ABSTRACT

Compositions and methods for large-scale recombinant protein expression are disclosed.

This application claims priority to U.S. Provisional Application No. 61/830,333 filed Jun. 3, 2013, the entire contents being incorporated herein by reference as though set forth in full.

This invention was made with US government support, NIH grant number R01 AI080659-01A2. Accordingly, the US government has rights in this invention.

### FIELD OF THE INVENTION

This invention relates to the fields of cell culture and large scale recombinant protein expression. More specifically, the invention provides methods and compositions which are effective to increase expression of recombinant proteins when compared to prior art methods.

### BACKGROUND OF THE INVENTION

Several publications and patent documents are cited throughout the specification in order to describe the state of the art to which this invention pertains. Each of these citations is incorporated herein by reference as though set forth in full.

Production of recombinant proteins relies heavily on expression systems developed in bacteria or lower eukaryotes (yeast, baculovirus, etc.). Many important human proteins have post-translational modifications (e.g. glycosylation and disulfide bonding) that make production in non-mammalian cells extremely challenging, since these expression systems fail to accurately replicate these modifications.

Previously, recombinant protein expression in mammalian cells was performed by transient transfection of suspension cells using the CaCl₂ method (1). Significant improvements in yield were achieved by utilizing polyethylenimine (PEI) to transiently introduce DNA expression plasmids (2). While these methods have remained the most common for large-scale protein production in mammalian cells, there are significant drawbacks. For example, protein expression levels slowly decrease since the DNA plasmids are not integrated into the host genome and are therefore lost during several rounds of cell division. If more protein is needed, the entire process must be repeated. Consequently, the expression is limited to a few weeks per round of transfection. Production of a stable expression cell line using DNA plasmids with an additional drug selection marker circumvents this problem but requires several weeks to establish the cell line. The major limitations with these methods are time, labor, and reagents.

Attempting to improve expression yields and feasibility, Aricescu et al published a description of HEK293 adherent cells transiently transfected using polyethylenimine (3). While they reported the recovery of 1-40 mg of protein per liter of media (4 roller bottles) amongst 24 targets, this method requires excessive plastics/disposable usage, which is both labor-intensive and costly. A review published in the same year describes the large-scale transfection of various suspension cell lines, outlining optimal transfection reagents, expression vectors, and media (4). Volumetric limitations and yield compromises are discussed and no significant improvement in the technology is reported. In 2009, Lee et. al. described a similar approach, where HEK293T cells were transfected with milligram quantities of DNA and grown in Corning CellStacks (5). Still, large amounts of DNA and cost-prohibitive transfection reagents and plastics make this approach impractical for many applications.

As can be seen from the above, conventional approaches to expressing recombinant proteins in mammalian cell lines is considered expensive, time consuming and not amenable to high throughput. Research in mammalian protein expression has progressed slowly over the last decade, owing to the high cost of media, serum, and disposable plastics, technically skilled personnel and facility requirements. For these reasons, mammalian cell culture is often viewed as a cumbersome undertaking.

### SUMMARY OF THE INVENTION

In accordance with the present invention, an improved culture system for the large scale production of recombinant proteins is provided. In one aspect of the invention, a lentiviral vector encoding a recombinant protein of interest is provided. In another aspect, a mammalian cell line comprising said lentiviral vector is also provided.

In a particularly preferred embodiment of the invention, a method for the large scale production of a recombinant protein of interest is disclosed. An exemplary method entails providing a lentivirus vector, a packaging vector and vector encoding VSV glycoprotein, said lentiviral vector encoding at least one recombinant protein of interest and infecting target mammalian cells with said vectors. Infected cells are then incubated in a bioreactor under conditions suitable for said recombinant protein to be produced and secreted from said infected cells. After a suitable time period, the secreted protein is harvested from the supernatant. The method may further entail introduction of kifunensine in media into said bioreactor. In a particularly preferred embodiment, the bioreactor is a BelloCell-500 bioreactor.

### BRIEF DESCRIPTION OF THE DRAWINGS

FIG. 1. The BelloStage3000 holding 3 BelloCell bioreactors.

FIG. 2. Coomassie stained SDS-PAGE gels comparing a transfected and stably selected plasmid clone (left, 100 ug/L) to the same clone integrated via lentivirus infection (right, 10 mg/L).

FIG. 3. A Coomassie stained SDS-PAGE gel providing a further example of the yields achieved. In this figure, a human scavenger receptor BI expressing at 50 mg/L is shown.

FIG. 4. A) A schematic representation of the pJG vector illustrates various elements, including those used for expression enhancement. B) A schematic representation of the pJG_euro vector.

### DETAILED DESCRIPTION OF THE INVENTION

We have developed a method that drastically improves the production of large quantities of recombinant proteins in mammalian cells. Our method combines the speed and high efficiency of lentiviral infection with an adherent cell bioreactor to allow large-scale production of human proteins in mammalian cell lines. This approach results in significant reductions in cost, while improving efficiency, quality, and utility for challenging and important classes of human proteins (e.g., those with specific post-translational modifications such as glycosylation and disulfide bonding). As a demonstration of solid proof of principle, this system has successfully and abundantly expressed viral and human proteins that have failed to express in any other system.

### Definitions

By “protein” or “polypeptide” is meant any chain of amino acids, regardless of length or post-translational modification. Proteins can exist as monomers or multimers, comprising two or more assembled polypeptide chains, fragments of proteins, polypeptides, oligopeptides, or peptides.

The term “a gene of interest” as used herein, refers to a nucleic acid sequence composed of one or more gene segments (genomic or cDNA) that encode one member of a protein of interest. The plural form “genes of interest” refers to a library of nucleic acid sequences encoding a polyclonal protein of interest. The term “GOI” is used as an abbreviation of (a) gene(s) of interest.

As used herein, the term “vector” refers to a nucleic acid molecule into which a nucleic acid sequence can be inserted for transport between different genetic environments and/or for expression in a host cell. If the vector carries regulatory elements for transcription of the nucleic acid sequence inserted in the vector (at least a suitable promoter), the vector is herein called “an expression vector”. If the nucleic acid sequence inserted into the above identified vectors encodes a protein of interest as herein defined, the following terms are used “vector of interest” and “expression vector of interest”.

The term “transfection” is herein used as a broad term for introducing foreign DNA into a cell. The term is also meant to cover other functional equivalent methods for introducing foreign DNA into a cell, such as e.g., transformation, infection, transduction or fusion of a donor cell and an acceptor cell.

The term “selection” is used to describe a method where cells have acquired a certain characteristic that enable the isolation from cells that have not acquired that characteristic. Such characteristics can be resistance to a cytotoxic agent or production of an essential nutrient, enzyme, or color.

The terms “selectable marker gene”, “selection marker gene”, “selection gene” and “marker gene” are used to describe a gene encoding a selectable marker (e.g., a gene conferring resistance against some cytotoxic drug such as certain antibiotics, a gene capable of producing an essential nutrient which can be depleted from the growth medium, a gene encoding an enzyme producing analyzable metabolites or a gene encoding a colored protein which for example can be sorted by FACS) which is co-introduced into the cells together with the gene(s) of interest.

The term “recombinant protein” is used to describe a protein that is expressed from a cell line transfected with an expression vector comprising the coding sequence of the protein.

As used herein, the term “operably linked” refers to a segment being linked to another segment when placed into a functional relationship with the other segment. For example, DNA encoding a signal sequence is operably linked to DNA encoding a polypeptide if it is expressed as a leader that participates in the transfer of the polypeptide to the endoplasmic reticulum. Also, a promoter or enhancer is operably linked to a coding sequence if it stimulates the transcription of the sequence.

The term “promoter” refers to a region of DNA involved in binding the RNA polymerase to initiate transcription.

The term “internal ribosome entry site” or “IRES” describes a structure different from the normal 5′ cap-structure on an mRNA. Both structures can be recognized by a ribosome to initiate scanning for an AUG codon to initiate translation. By using one promoter sequence and two initiating AUG's, a first and a second polypeptide sequence can be translated from a single mRNA. Thus, to enable co-translation of a first and a second polynucleotide sequence from a single bi-cistronic mRNA, the first and second polynucleotide sequence can be transcriptionally fused via a linker sequence including an IRES sequence that enables translation of the polynucleotide sequence downstream of the IRES sequence. In this case, a transcribed bi-cistronic RNA molecule will be translated from both the capped 5′ end and from the internal IRES sequence of the bi-cistronic RNA molecule to thereby produce both the first and the second polypeptide.

The term “inducible expression” is used to describe expression that requires interaction of an inducer molecule or the release of a co-repressor molecule and a regulatory protein for expression to take place.

The term “constitutive expression” refers to expression which is not usually inducible.

The methods set forth below are provided to facilitate the practice of the present invention.

### Methods

1. Cloning. Signal sequences and expression tags are important considerations when optimizing recombinant proteins for secretion in mammalian cells. We have chosen a protein-A repeat tag which binds to a recombinant IgG column with high affinity, has no cysteine residues, and is glycosylated. This provides an efficient method to assist the protein through the correct folding pathway while enabling the purification of secreted protein from cell culture supernatant. The signal sequence and protein-A tag are fused to the protein of interest in a sub-cloning procedure prior to amplification for pJG (lentiviral vector) insertion. This allows preliminary expression testing via transient transfection and facilitates cloning into pJG. In circumstances where no antibody is available for detection of the protein of interest, a 3X-Flag tag (Sigma) is fused to the protein-A tag in tandem. The pJG vector is digested using the PmeI restriction enzyme, followed by treatment with CIP to prevent re-ligation. The gene of interest (including the signal sequence and tag) is amplified using PmeI complimentary forward and reverse primers, with InFusion-HD (Clontech) overhangs. InFusion-HD cloning is executed according to the manufacturer's instructions and the reaction is transformed into HB101 cells. The DNA is purified using the Hispeed Plasmid Purification Kit from Qiagen for optimum yield and minimal ethanol contamination.

2. Lentiviral production.

a) Seeding. One day prior to planned transfection, a single T-225 monolayer flask is seeded with 6.0×10⁶ HEK293T cells.

b) Transfection. The co-transfection protocol for these vectors is done via the CaCl₂ method with HEPES buffered saline. Combined in a 50 mL Falcon tube: 90 ug pJG-GOI, 60 ug psPAX2, 30 ug pMD2.G, 450 uL CaCl₂, q.s. to 4.5 mL of ddH₂0. 4.5 mL of room temperature 2× HEPES buffer is added to the mixture and bubbled with a serological pipet for 10 seconds. The mixture is incubated at room temperature for 2 minutes and is then added directly to the culture media in the flask prepared as per 2.b. 6-8 hours later, the media is aspirated from the cells and replaced with 40 mL of fresh media.

c) Preparing for infection. Two days after the transfection, 10,000 cells are seeded into a single well of a 96-well plate in a final volume of 50 uL (2×10⁵ cells/mL).

d) Harvesting virus. The supernatant from the transfection, containing the recombinant lentiviruses, is harvested and centrifuged for 30 mins at 4,000×g at 4° C. to pellet major cellular debris. 37 mL of clarified supernatant is transferred to a Beckman Ultracentrifuge tube fitted for an SW28 rotor. Virus is then pelleted for 1.5 hr at 25,000 rpm (80,000×g) at 4° C. using maximum acceleration and deceleration. Supernatant is decanted into a waste container filled with 1% vesphene. The pellet is dried, inverted, for 5 minutes, then resuspended in 120 uL of: DMEM+20% FBS+1% antibiotic/antimycotic (A/A)+8 ug/mL polybrene.

e) Infection. As described above, one well of a 96-well plate was seeded with 10,000 cells one day prior to harvesting virus; the media can now be aspirated from this well and replaced with 5 0uL of virus suspension for overnight incubation. (The remaining 70 uL can be frozen and stored at −20 ° C.). The following morning, 50 uL of fresh DMEM+10% FBS+1% A/A is added to the infected well. On the third day, the media is removed and replaced with 100 uL of fresh DMEM+10% FBS+1% A/A. At this time, a view of GFP expression will indicate the approximate efficiency of infection. Cell expansion can begin as soon as confluence is reached.

3. Bioreactor Maintenance. A single BelloCell-500 bioreactor is typically seeded with ˜5.0-8.0×10⁷ HEK293T cells, the equivalent of 3-4 confluent T-175 monolayer flasks. The seeding protocol is followed in accordance with the manufacturer's instructions, with an oscillation rate of 2.0 mm/s up and down, a top hold time of 20 sec, and a down hold time of 0 sec. After 2-4 hours using the seeding protocol, the oscillation speed is reduced to 1.0 mm/s, with a top hold time of 10 sec and a bottom hold time of 1 min.

a) Harvesting secreted protein. Approximately 3 days after seeding, or when the glucose reading for the media falls below 1 g/L, the media is aspirated from the bottom chamber and refreshed with 500 mL of fresh media. After this, the media is harvested and replaced every two days. The addition of kifunensine to the media at 1 mg/L does not affect the harvesting schedule.

b) Harvesting intracellular protein. The BelloCell-500D utilizes the same bioreactor design with a digestable matrix. The bioreactor is maintained and harvested as described in 1.a., with the addition of a maintenance reagent required for this model. Within 3 weeks, the fully populated matrix can be digested according to the manufacturer's protocol, yielding an approximate cell count of at least 1×10⁹ cells/mL. Cells are lysed via sonication.

The following Example is provided to illustrate certain embodiments of the invention. It is not intended to limit the invention in any way.

### Example I

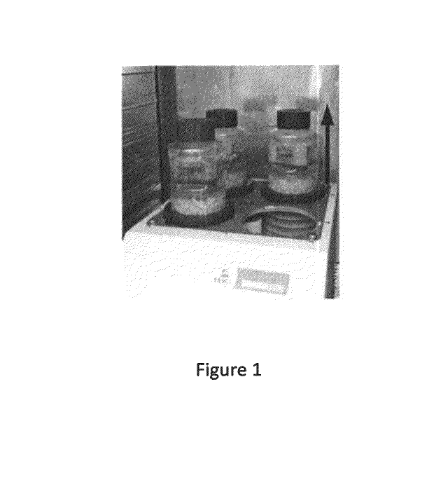

To address the ongoing challenges associated with recombinant protein expression in mammalian cells, the system detailed below integrates several different elements of cell culture technology for a synergistic result. The BelloCell-500 bioreactor (Cesco Bioengineering, Taiwan) was chosen for its ideal investment/output ratio. Its innovative design addresses several adherent cell culture challenges (6). Relative to the systems described above, the BelloCell: a) consumes significantly less disposable products and waste, resulting in a substantial cost saving of almost 50%; b) utilizes a three dimensional attachment matrix, allowing for better use of space and a higher degree of cell density; and c) provides a more ideal growth environment in terms of temperature distribution and gas exchange. The BelloCell operates on a programmable stage called the BelloStage 3000, which holds 4 bioreactors and fits inside a standard cell culture incubator (FIG. 1). The bottle is divided into two chambers: the top chamber houses a polyester matrix onto which adherent cells attach and the bottom chamber is a flexible bellows, which holds 500 mL of media. During the upward oscillation, media flows over the matrix to feed the cells. During the downward oscillation, media is contained within the bellows, allowing gas exchange. The top hold time and bottom hold time can be adjusted for seeding, cell type, and growth rate control. In our hands, a single bioreactor can be maintained for at least 3 months with continuous media harvesting and replacement. We have demonstrated successful protein expression using both HEK293T and HeLa cells, resulting in as much as a 10-fold increase in protein production over conventional roller bottles for the same cell lines.

The second component contributing to the efficiency of this system is the use of lentiviral expression vectors. Lentiviral vectors have emerged over the last decade as powerful, reliable, and safe tools for stable gene transfer in a wide variety of mammalian cells (7). In addition, lentiviral expression systems provide a cost and labor effective method of generating stably expressing cell lines within a short period of time on the order of days to a week. Lentiviruses are a genus of the Retroviridea family that can efficiently and conveniently infect a wide variety of mammalian cells, including non-dividing cells. Upon infection, the viral RNA genome is reverse-transcribed into DNA, which is transported to the nucleus and integrated into the genome by a viral-encoded integrase enzyme. Lentiviruses have been adapted to safely integrate genetic material into the cellular chromosomes, creating a stable cell line. The use of multiple plasmids prevents recombination of genetic material to produce an intact viral genome, resulting in a pseudotype lentivirus capable of only a single round of infection into a variety of mammalian cell lines. The retroviral envelope glycoproteins have been replaced with the G protein of vesicular stomatitis virus (VSV-G) to allow for greater host cell infection and added stability. The entire process of stable cell line production using lentiviral vectors can be performed in a week, bypassing the time consuming procedure of drug selection. The lentiviral system is more rapid and efficient than previous approaches because it circumvents the needs to: 1) use special media or drugs for selection in cell culture, 2) isolate clonal cell lines, and 3) screen large numbers of cell lines to find a high producer. As a result the generation time to produce a stable cell line significantly shortens from 2-3 months to about one week.

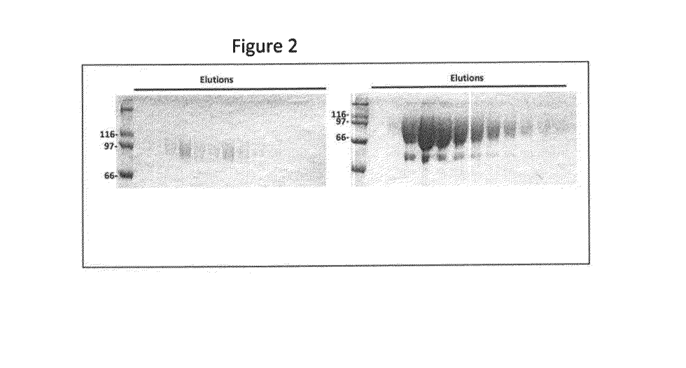

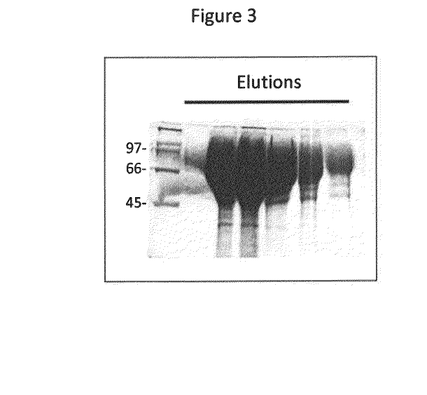

In addition to efficiency, typically lentivirus expression also results in a substantial increase of protein yield owing to the high copy number of expression cassettes. The number of copies of the expression cassette introduced into a cell is correlated to the number of virus particles entering the cell, or multiplicity of infection (MOI). Thus, protocols can be designed such that all cells are infected at extremely high MOIs (100-1,000), eliminating the need to culture cells in selective media to identify clones that contain the expression cassette. FIG. 2 illustrates an approximate 100-fold increase in expression for lentiviral infected cells (right) versus the original transfection/stable selected cell clone (left). In addition, FIG. 3 provides a further example of the yields achieved, a human scavenger receptor BI expressing at 50 mg/L.

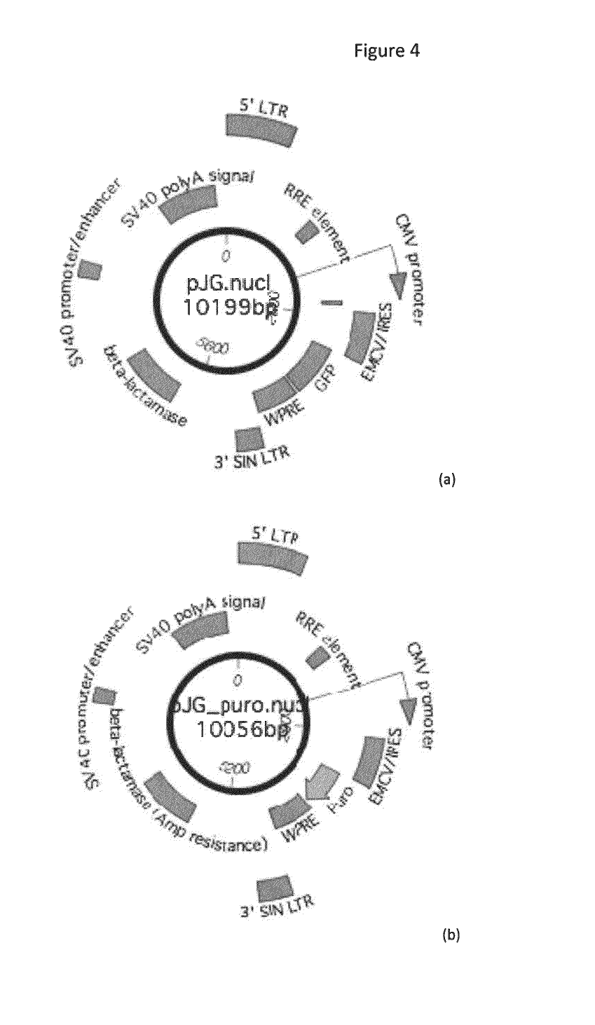

Originally described by Trono et.al, two components of the lentiviral system described here are currently available through AddGene.⁷ pMD2.G encodes VSV glycoproteins for incorporation into the viral envelope and versatility of cell tropism, and psPAX2 contains the standard HIV Gag/Pol cassette. The third plasmid, pJG, was reengineered to further improve viral titres. pJG uses a CMV promoter to drive expression, with additional enhancement provided by the Rev Response Element and a Woodchuck Hepatitis promoter enhancer element (FIG. 4).⁷ In both its current and original design, this vector contains a GFP gene immediately following the cloned gene, separated by an IRES. This provides an early visible marker for infection efficiency, which correlates to the expression of the protein of interest.⁸ Another version of pJG in which the GFP gene is replaced with a puromycin resistance gene has been created, allowing for the selection of puromycin resistant cells, further amplifying protein production.

Finally, the heterogeneity of post-translational modifications can be problematic for certain downstream protein applications. Therefore, the cell culture media is supplemented with mannoside inhibitors, producing more homogenized glycosylation. Kifunensine, a mannosidase inhibitor first described in 1990, is used to prevent complex and hybrid glycosylation from building throughout the secretory pathway.⁹ The effectiveness and expression results with the use of kifunensine have been thoroughly described.¹⁰ This compound works at low effective concentrations and conveniently increases protein yields by approximately 2-fold, although the mechanism of this enhancement is not clear.

The compositions and methods described herein are useful for the large scale production of recombinant proteins and thus provide a valuable tool for production and biochemical characterization of the same.

### Example II

In further experiments, we have used the system described in Example I to express abundant amounts of hepatitis C envelope glycoprotein 2 and human CD81-LEL. These methods are set forth below.

HCV J6 eE2 Expression using the Mammalian Protein Expression System of the Invention

eE2, eE2(ΔHVR1) and E2 core domain encompasses residues 384-656, 413-656 and 456-656 from the HCV J6 genome, respectively. Owing to incomplete deglycosylation at N7 (542) with EndoH, the crystallization construct contained an asparagine to glutamine mutation at this position. The expression constructs consisted of a CMV promoter, a prolactin signal sequence, E2 fragment, PreScission Protease cleavage site and a C-terminal protein-A (ProtA) tag. The entire prolactin-E2-ProtA sequence was PCR amplified and cloned into the pJG lentiviral vector described in Example I.

Wild-type and GnTI-HEK293T cells were maintained in Dulbecco's Modified Eagle Medium (DMEM) with 10% fetal bovine serum (FBS) at 37° C. with 5% CO₂. One day before the planned transfection, a single T-225 monolayer flask was seeded with 6.0×10⁶ HEK293T cells. 90 μg pJG-E2, 60 μg psPAX2 (HIV Gag-Pol packaging vector), 30 μg pMD2.G (VSV glycoprotein vector) and 450 μl of 2 M CaCl₂ were mixed and brought to a final volume of 4.5 ml with ddH₂0. 4.5 ml of 2×HEPES buffered saline was added at room temperature. After a 2-min incubation, the mixture was added directly to HEK293T cells. After 6-8 h, the media was replaced with DMEM with 10% FBS and 1% antibiotic/antimycotic (A/A) media and incubated for another 70 h.

Two days after transfection, 10,000 GnTI-HEK293T cells were seeded into a single well of a 96-well plate. The supernatant from the transfection, containing the recombinant lentiviruses, was collected and centrifuged for 30 min at 4,000 g at 4° C. to pellet large cellular debris. Clarified supernatant was transferred to a Beckman Ultracentrifuge tube and virus was pelleted for 1.5 h at 25,000 r.p.m. (80,000 g) at 4° C. in an SW28 rotor. Supernatant was discarded and the pellet re-suspended in 120 μl of DMEM containing 20% FBS, 1% A/A, and 8 μg ml⁻¹ of polybrene. 60 μl⁻¹ of virus suspension was added to the prepared GnTI-HEK293T cells and incubated overnight. Infected cells were expanded and ultimately seeded into an adherent cell bioreactor (Cesco Bioengineering) for long-term growth and protein production.

### CD81 Purification and Binding Assays

Human CD81-LEL (residues 112-202) was produced as a fusion with C-terminal ProtA tag in HEK293T cells using the same lentiviral expression system described for eE2. Cell culture supernatants were loaded onto an IgG FF column, washed with 20 mM sodium phosphate pH 7.0, eluted with 100 mM sodium citrate pH 3.0 containing 20 mM KCl and immediately neutralized with 1 M Tris pH 9.0. The ProtA tag was cleaved by PreScission Protease (GE Healthcare Life Sciences) in a ratio of 1:50 (w/w) followed by overnight dialysis in 20 mM HEPES pH 7.5, 250 mM NaCl, and 5% glycerol. High-purity CD81 protein was obtained by anion exchange and size-exclusion chromatography.

For binding studies, a 96-well plate (Nalgene Nunc, Thermo Fisher Scientific) was coated with 50 μg of CD81-LEL overnight at 4° C. All experiments were duplicated against BSA as a negative control. Plates were washed three times with PBS containing 0.05% Tween 20 (PBS-T) and blocked with 3% (w/v) BSA in PBS-T for 1 h at room temperature. 50 μl of eE2 or E2 core at different concentrations was added to appropriate wells and incubated overnight at 4° C. On day 3, the wells were washed three times with PBS-T and incubated with monoclonal antibody 2A12 cell supernatant for 1 h at room temperature. Plates were washed three times with PBS-T and incubated with anti-mouse-HRP conjugated antibody for 1 h at room temperature. Finally, the plate was washed five times with PBS-T. 50 μl of TMB substrate (ThermoFisher Scientific) was added to each well and incubated for 5 min, followed by the addition of 50 μl of 2 M sulphuric acid to stop the reaction. Absorbance readings were acquired at 450 nm using Softmax Pro software on a Spectra Max 250 (Molecular Devices).

### REFERENCES

1. Jordan M, Kohne,C., Wurm, F. M. Calcium-phosphate mediated DNA transfer into HEK-293 cells in suspension: control of physiological parameters allows transfection in stirred media. Cytotechnology 1998;26:29-47.

2. Schlaeger E J, Christensen K. Transient gene expression in mammalian cells grown in serum-free suspension culture. Cytotechnology 1999;30:71-83.

3. Aricescu A R, Lu W, Jones E Y. A time- and cost-efficient system for high-level protein production in mammalian cells. Acta Crystallogr D Biol Crystallogr 2006;62:1243-50.

4. Pham PL, Kamen A, Durocher Y. Large-scale transfection of mammalian cells for the fast production of recombinant protein. Mol Biotechnol 2006;34:225-37.

5. Lee J E, Fusco M L, Saphire E O. An efficient platform for screening expression and crystallization of glycoproteins produced in human cells. Nat Protoc 2009;4:592-604.

6. Wang I K, Hsieh S Y, Chang K M, et al. A novel control scheme for inducing angiostatin-human IgG fusion protein production using recombinant CHO cells in a oscillating bioreactor. J Biotechnol 2006;121:418-28.

7. Naldini L, Blomer U, Gallay P, et al. In vivo gene delivery and stable transduction of nondividing cells by a lentiviral vector. Science 1996;272:263-7.

8. Mancia F, Patel S D, Rajala M W, et al. Optimization of protein production in mammalian cells with a coexpressed fluorescent marker. Structure 2004;12:1355-60.

9. Elbein A D, Tropea J E, Mitchell M, Kaushal G P. Kifunensine, a potent inhibitor of the glycoprotein processing mannosidase I. J Biol Chem 1990;265:15599-605.

10. Chang V T, Crispin M, Aricescu A R, et al. Glycoprotein structural genomics: solving the glycosylation problem. Structure 2007;15:267-73.

While certain preferred embodiments of the present invention have been described and specifically exemplified above, it is not intended that the invention be limited to such embodiments. Various modifications may be made to the invention without departing from the scope and spirit thereof as set forth in the following claims.

What is claimed is:

1. A lentiviral vector comprising a nucleic acid encoding a recombinant protein of interest.

2. The lentiviral vector of claim 1, which is a pJG lentiviral vector comprising a promoter sequence, a signal sequence, a sequence encoding a protein of interest, a cleavage sequence and a C-terminal tag sequence.

3. A cell line comprising a vector as claimed in claim 2.

4. A method for the large scale production of a recombinant protein of interest comprising;
a) providing a lentivirus vector as claimed in claim 2, a packaging vector and a vector expressing VSV glycoprotein;
b) infecting target mammalian cells with said vectors and incubating said cells in a bioreactor under conditions suitable for said recombinant protein to be produced and secreted from said infected cells, and
c) harvesting said secreted protein.

5. The method of claim 4 comprising introduction of kifunensine in media into said bioreactor.

6. The method of claim 5, wherein said bioreactor is a BelloCell-500 bioreactor.

7. The method of claim 6, wherein said protein is selected from the group consisting of HCV E2 core protein and CD81.
